(19) United States
(12) Patent Application Publication
(10) Pub. No.: US 2002/0129989 A1
(43) Pub. Date: Sep. 19, 2002

(54) Stethoscope sound isolation headset

(76) Inventor: Mark Parsons, Hollywood, FL (US)

Correspondence Address: David P. Lhota, Stearns Weaver Miller, et al., Suite 1900, 200 East Broward Boulevard, Fort Lauderdale, FL, 33301, US

(21) Appl. No.: 10/082,775

(22) Filed: Feb. 25, 2002

Related U.S. Application Data
(63) Continuation-in-part of application No. 09/614,283, filed on Jul. 12, 2000.

(51) Int. Cl. A61B 7/02

(52) U.S. Cl. 181/131; 181/130

(57) ABSTRACT

A stethoscope amplifier headset for receiving, isolating and transferring sound received from a stethoscope to a user's ears, the stethoscope sound isolation headset including left and right ear assemblies connected by an adjustable length headband, ear receiving cavities, stethoscope receiving apertures, and acoustic insulators. Each of the left and right ear assemblies may also include an amplifier assembly including an adjustable volume control.

### CROSS REFERENCE TO RELATED APPLICATIONS

This application is a continuation-in-part of U.S. application Ser. No. 09/614,283 filed Jul. 12, 2000.

### STATEMENT REGARDING FEDERALLY SPONSORED RESEARCH OR DEVELOPMENT

N/A

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office patent file or records, but otherwise reserves all copyrights rights whatsoever.

### BACKGROUND OF THE INVENTION

1. Field of the Invention

The present invention relates generally to headsets, and more particularly, to a headset that is adapted for use with a stethoscope to isolate sound received through the stethoscope ear pieces.

2. Description of the Background Art

Physicians, emergency personnel, paramedics and other healthcare workers use stethoscopes to listen to faint sounds generated by operation of certain organs such as the lungs and heart. Stethoscopes convey sounds generated by internal bodily vibrations through tubes and earplug outputs to the attendant's ear. These sounds are especially difficult to hear in noisy environments such as accident scenes and emergency rooms. It would be a benefit to healthcare and emergency personnel to have a stethoscope headset that could isolate sounds detected by the stethoscope from exterior noises. This would enhance the healthcare providers ability to more effectively utilize the stethoscope. As some bodily sounds may require amplification, it would be a further benefit to have a stethoscope amplifier headset that included an amplifier. In addition, an amplifier headset that had the ability to be selectively activate and deactivated would allow its user to switch the headset off so that some ambient sounds could be heard, such as an assistant's voice.

Stethoscope headset systems are generally known in the background art. However, none of these addresses or solves the problems noted in the prior art. For instance, U.S. Pat. No. 5,604,811, issued to McIntyre et al., discloses a stethoscope and headset system comprising a stethoscope having a monitor mechanism for monitoring internal bodily vibrations of a patient and conversion circuitry coupled to the monitor mechanism for receiving and transmitting internal bodily sounds, headband, microphone for receiving a user's voice and transmitting intercom signals, speakers, and switch for listening to a patients bodily sounds or connecting to an aircraft's intercom system. U.S. Pat. No. 4,299,303, issued to Clark, discloses a noise attenuating stethoscope coupled to a noise attenuating headset, resilient sealing device positioned in the ear cups, and quick-connect couplings between sound pick up tubes and sound delivery tubes.

### BRIEF SUMMARY OF THE INVENTION

Based on the foregoing, it is an object of the instant invention to provide a stethoscope sound isolation headset that isolates sounds detected by the stethoscope from other sounds, such as ambient noise.

It is another object of the instant invention to provide a stethoscope sound isolation headset that compartmentalizes and conveys sounds at the exclusion of undesirable sounds.

It is also object of the instant invention to provide a stethoscope sound isolation headset that can be selectively activated and deactivated.

It is additional object of the instant invention to provide a stethoscope sound isolation headset that is convenient to use and cost-effective in structure.

It is a further object of the instant invention to provide a stethoscope sound isolation headset that mechanically transmits and directs bodily sounds detected by the stethoscope.

It is yet another object of the instant invention to provide a stethoscope sound isolation headset that can amplify detected by the stethoscope.

It is a further object of the instant invention to provide a stethoscope sound isolation headset that mechanically transmits and directs bodily sounds detected by the stethoscope.

In light of these and other objects, the instant invention comprises a stethoscope sound isolation headset, generally referenced herein as a stethoscope headset, that is adaptable for use with a stethoscope and isolates and directs body generated sounds to the cognizant attendant. The instant invention may further include a stethoscope. The stethoscope headset comprises left and right ear assemblies that receive, capture and transfer sound detected by the stethoscope to the user's ears substantially to the exclusion of ambient noise. The ear assemblies are preferably coupled together by a flexible, length-adjustable head band and a removable, adjustable and rotatable rigid head band. The ear assemblies each include a molded ear cover member having the general shape of a bowl so as to provide an ear receiving cavity formed by an interior surface thereof, stethoscope coupling cavity for receiving the stethoscope ear adaptors, rubber acoustic/sound insulator, rotatable head band adapter and soft, pliable gasket along the circumferential edge of the ear cover member for providing a sealed fit over the ear to prevent acoustic leaks. The ear cover members may also have a foam acoustic insulator to enhance the sound barrier characteristics of the acoustic insulators. The foam acoustic insulator and, or rubber acoustic insulator are preferably secured in the ear receiving cavity by an adhesive. A narrow, self-sealing slit is defined in the rubber acoustic insulator for receiving and passing the stethoscope ear adaptors. The rubber acoustic insulator may be further secured by a support plate, which is secured over the rubber insulator on the interior surface of the ear-receiving cavity. The support plate includes an aperture that is concentrically aligned with the rubber insulator slit for facilitating the passage of the stethoscope ear adaptors. In an alternative embodiment, the ear cover members may have an ambient sound-receiving aperture that may be selectively opened and closed with a mechanical knob disposed on an exterior surface.

In another alternative embodiment, the ear cover members may be formed from an acoustic insulating material into the general shape of a bowl to define the ear receiving cavity, speaker cavity formed into the interior surface inside the ear receiving cavity and microphone coupling cavity formed into an exterior surface of the of the acoustic insulation material. The speaker cavity is in acoustical communication with the ear-receiving cavity. In an alternative embodiment, the ear cover member may also include an amplifier assembly for amplifying sound received from the stethoscope. The amplifier assembly comprises an amplifier cavity formed within the acoustic insulating material and may also include an amplifier therein. The amplifier may comprise a mechanical amplifier or an amplifier circuit positioned within the amplifier circuit cavity. The invention may also include a microphone having an acoustical input in connection with a stethoscope coupling cavity, speaker installed within the speaker cavity having a speaker output in acoustical connection with the ear receiving cavity, and on-off knob for selectively activating and deactivating the headset. In the embodiment incorporating an amplifier, the invention may further include a volume adjustment knob mounted to the exterior facing side of the ear cover member for adjusting the amplification of the amplifier. The amplifier circuit preferably includes a microphone input in electrical connection with the microphone, a speaker output in electrical connection with the speaker, and an amplifier volume control input in electrical connection with the volume adjustment knob. The microphone-coupling cavity is acoustically isolated from the speaker cavity by a portion of the ear cover member's acoustic insulating material to effectuate proper sound isolation.

In accordance with these and other objects, which will become apparent hereinafter, the instant invention will now be described with particular reference to the accompanying drawings.

### BRIEF DESCRIPTION OF THE SEVERAL VIEWS OF THE DRAWINGS

For a further understanding of the nature and objects of the present invention, reference should be made to the following detailed description, taken in conjunction with the accompanying drawings, in which like elements are given the same or analogous reference numbers and wherein:

FIG. 1 is a front elevational view of the preferred embodiment of the stethoscope headset and stethoscope of the present invention.

FIG. 2 is a front exploded view of the preferred embodiment of the stethoscope headset and stethoscope of the present invention.

FIG. 3A is an interior side elevational view of the preferred embodiment of the ear cover members of the present invention.

FIG. 3B is an exterior side elevational view of the preferred embodiment of the ear cover members of the present invention.

FIG. 4A is a side perspective view of the preferred embodiment of the ear cover members of the present invention illustrating the rigid headband in a first upright position.

FIG. 4B is a side perspective view of the preferred embodiment of the ear cover members of the present invention illustrating the rigid headband in a second position.

FIG. 5 is an elevational exploded view of an alternative embodiment of the stethoscope headset of the present invention.

FIG. 6 is a pictorial elevational view of the instant invention illustrating use of the instant invention.

FIG. 7 is a cross section view of an alternative embodiment of the left and right ear assemblies in accordance with the instant invention.

### DETAILED DESCRIPTION OF THE INVENTION

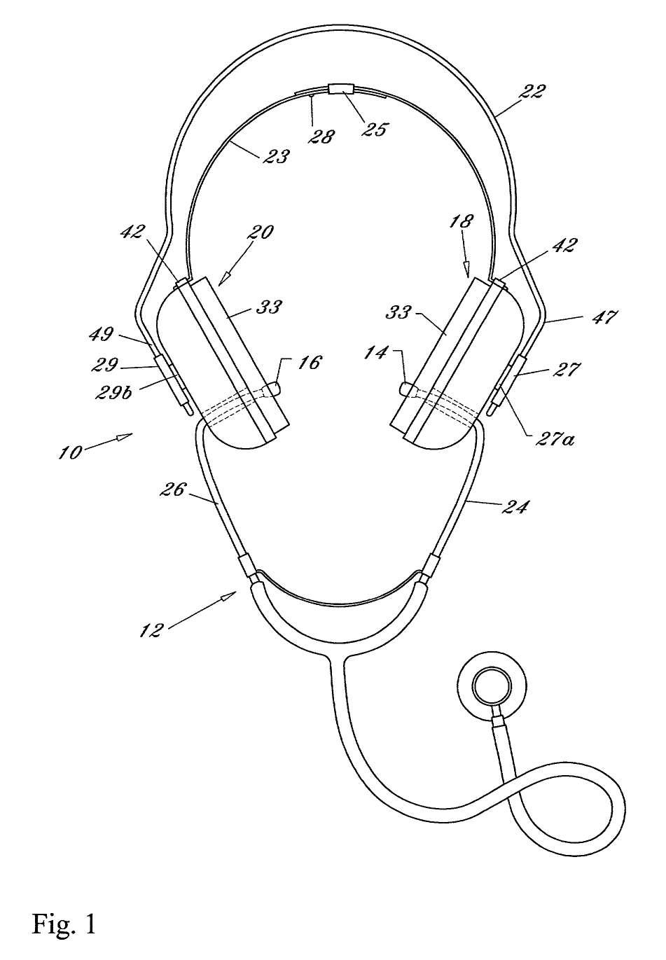

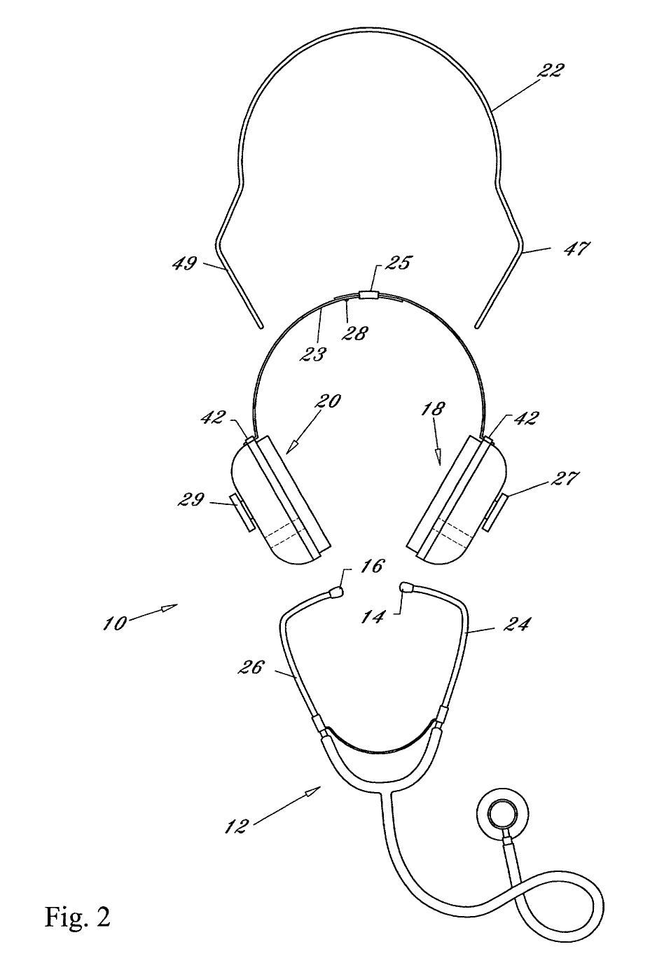

With reference to the drawings, FIGS. 1-7 depict the preferred and alternative embodiments of the instant invention, which is generally referenced as a stethoscope headset and, or by reference numeral 10. Referring to FIGS. 1 and 2, the instant invention 10 comprises a stethoscope sound isolation headset that is adaptable for use with a stethoscope and isolates and directs body-generated sounds to the cognizant attendant. The instant invention may further include a stethoscope 12. The stethoscope headset comprises left and right ear assemblies 18, 20 that receive, capture and transfer sound detected by the stethoscope to the user's ears substantially to the exclusion of ambient noise. The ear assemblies 18, 20 are coupled together by a flexible, length-adjustable head band 23 and removable, adjustable and rotatable rigid head band 22.

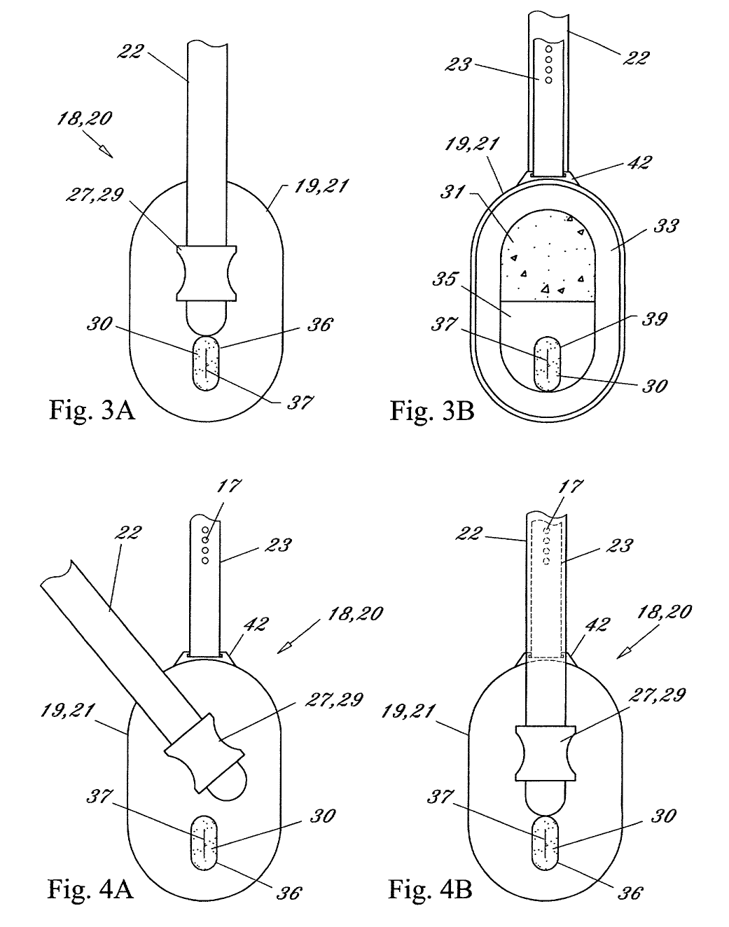

With reference to FIGS. 3A and 3B, the ear assemblies 18, 19 each comprise a molded ear cover member 19, 21, respectively, having the general shape of a bowl so as to define an ear receiving cavity 34 along an interior surface thereof, stethoscope coupling cavity 36 for receiving the stethoscope ear adaptors 14, 16, rubber acoustic/sound insulator 30, rotatable head band adapter 29 and soft, pliable gasket 33 secured along the circumferential edge of each ear cover member 19, 20 for providing a sealed fit over the wearer's ear to prevent acoustic leaks. The ear-receiving cavity captures, isolates and transfers sounds received from the stethoscope's sound transporting tubes 24, 26 The cavity 34 may also act as a mechanical amplifier. The ear cover members 19, 21 may also have a foam acoustic insulator 31 above and contiguous with the rubber insulator 30 to enhance the sound barrier characteristics of the instant invention. The ear cover members 18, 20 preferably comprise a rigid, durable hard plastic shell and define the stethoscope coupling cavities 36. The stethoscope coupling cavities 36 comprise apertures cut into the ear cover members 19, 21 in a strategically selected position for aligning the stethoscope adaptors 14, 16 with the wearer's ears. The foam acoustic insulator 31 and, or rubber acoustic insulator 30 are preferably secured in the ear receiving cavity 34 by an adhesive. The rubber acoustic insulator 30 comprises a resilient, flexible pad that completely covers the opening of the stethoscope-coupling cavity 36 so as to be substantially devoid of acoustic leaks. A narrow, elongated, self-sealing slit 37 is defined in the rubber acoustic insulator 30 for flushly receiving and passing the stethoscope ear adaptors 14, 16. The slit 37 is elongated for adjustment of the stethoscope ear adaptors 14, 16. The rubber acoustic insulator 30 may be further secured by a steel or plastic support plate 35, which is secured over the rubber insulator 30 on the interior surface of the ear-receiving cavity 34. The support plate 35 includes an aperture 39 that is concentrically aligned with the rubber insulator slit 37 for facilitating the passage of the stethoscope ear adaptors 14, 16 through the ear cavities 34. The support plate 35 is preferably adhered to the interior surface of the ear cover members 19, 21, but may also be secured with fasteners such as screws or rivets.

With reference to FIGS. 4A and 4B, the stethoscope headset 10 has adjustable headbands 22 and 23. The rigid headband 22 comprises a hard plastic and is adjustably supported by the first and second headband support adaptors 27, 29. The rigid headband 22 may have an elongated slot with a plurality of crescent shaped grooves for securing the headband in a desired position and facilitating height adjustment. The headband adaptors 27, 29 are secured to the exterior surface of the ear cover members 19, 21, respectively. The headband adapters 27, 29 have sleeves adapted for slidably receiving and securing the rigid headband's 22 opposing first and second support ends 47,49. The sleeves have a size that preferably accommodates a friction fit with the first and second support ends 47, 49 for securing them in place. The sleeves also having an interiorly disposed tab for engaging the crescent shaped grooves. The height of the rigid headband 22 is adjusted by sliding it through the headband support adaptor's 27, 29 sleeves with sufficient force to overcome the friction force. The adaptors 27, 29 have shafts 27a, 29a, respectively, that penetrate the ear cover members 19, 21 and an interior knob for preventing the shaft from being pulled out. The adaptors 27, 29 rotate about the shafts 27a, 29a, respectively, when adjusting the angle of the rigid headband 22. The headband 22 may be adjust at a plurality of different angles and may be completely removed. The flexible headband 23 is secured at its end to a closed hook 42 and have enlarged ends for preventing the flexible headband 23 from being inadvertently removed. The flexible headband 23 is joined together at its midsection by a clip 25 that has at least one protruding tab 28. The flexible headband 23 has a plurality of apertures 17 for receiving the tab 28 when adjusting and securing the length of the headband 23. The flexible headband 23 allows the headset 10 to be hung around a user's neck when not in use.

In an alternative embodiment, the ear cover members 19, 21 may have an ambient sound-receiving aperture 41 that may be selectively opened and closed with a mechanical knob 43 disposed on an exterior surface. The knob 43 actuates a slidable door 45 that resides over the aperture 41. The door 45 may be opened for hearing outside voices or other ambient sounds.

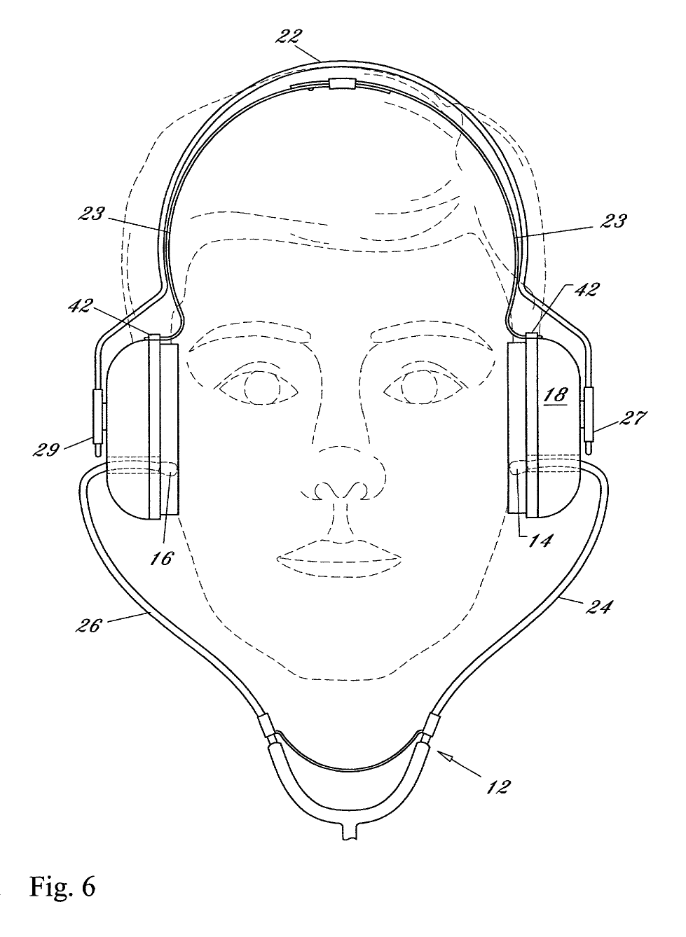

To use the stethoscope headset 10, the stethoscope ear adapters 14, 16 are inserted into the slits 37 in the respective ear cover members 19, 21. The slit 37 and rubber insulator 30 form around the stethoscope sound transfer tubes 24, 26. With reference to FIG. 6, the headset 10 is then placed on the users head and the ear adaptors into the user's ears. Sound from the stethoscope 12 is transported into the ear-receiving cavity 34, where it is isolated and transferred to the user's ears. The rigid headband 22 may be adjusted for comfort or completely removed. The flexible headband 23 supports the stethoscope headset 10 on the wearer and allows the headset 10 to be worn around a user's neck. In the alternative embodiment employing a knob 43, door 45 and ambient sound-receiving aperture 46, ambient sound may be heard by turning the knob 43 to open the door 45 for revealing the sound-receiving aperture 46. Ambient noise is blocked out by turning the knob 43 in a manner that closes the door 45.

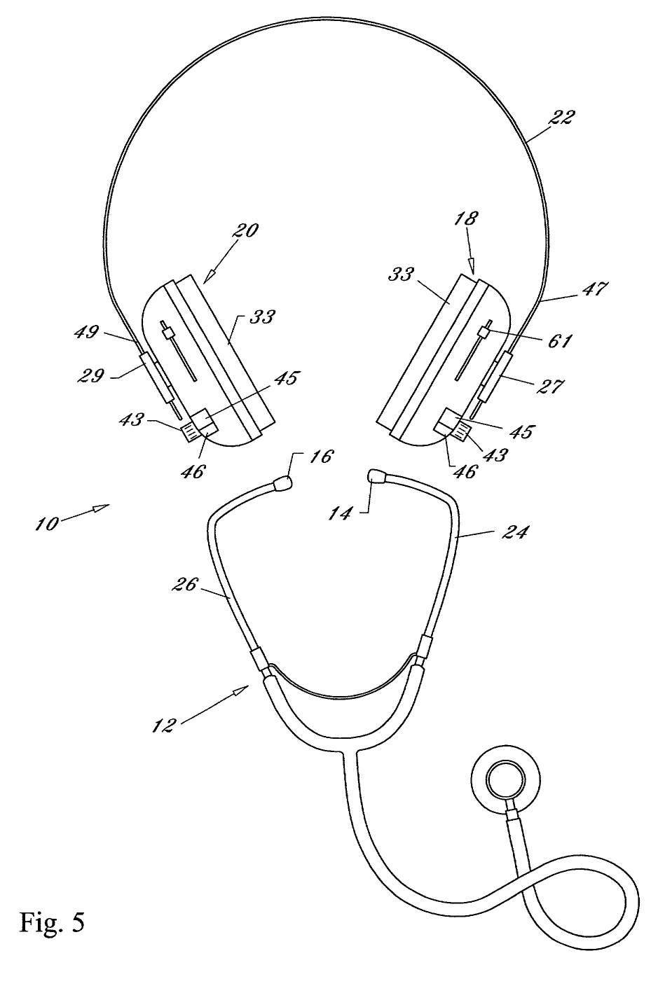

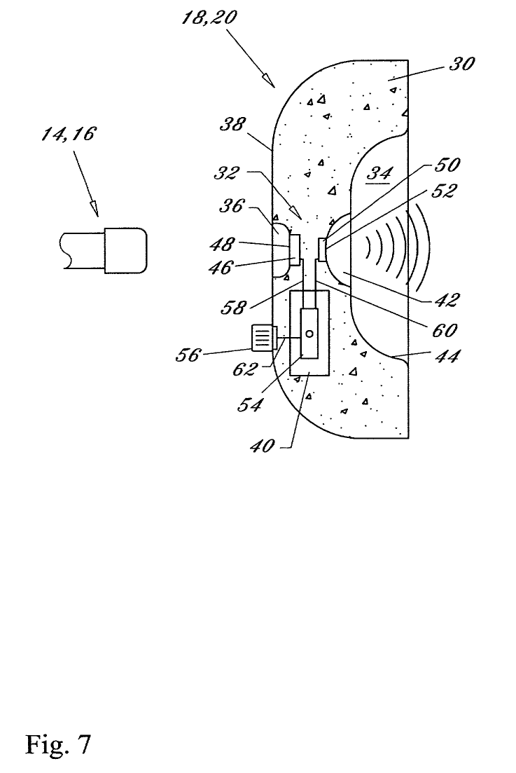

With reference to FIGS. 5 and 7, an alternative embodiment of the stethoscope headset 10 includes a left ear assembly 18, right ear assembly 20 and an adjustable length headband 22. The left ear assembly 18 and right ear assembly 20 are coupled to the headband 22. In the alternative embodiment, the ear cover members 19, 21 may be molded from an acoustic insulating material, such as 30 or 31, into a bowl shape that defines an ear receiving cavity 34. The alternative embodiment further comprises a stethoscope coupling cavity 36 formed in the exterior surface of the ear cover members 19, 21, amplifier circuit cavity 40 formed entirely therein, and speaker cavity 42 formed in and in acoustical connection with the ear receiving cavity 34. The amplifier assembly includes an amplifier circuit 54 positioned within the amplifier circuit cavity 40, microphone 46 having an acoustical input in communication with the stethoscope coupling cavity 36, speaker 50 installed within the speaker cavity 42 having a speaker output in acoustical connection with the ear receiving cavity 34, and volume adjustment knob 56 or 61 moveably positionable and mounted to the exterior of the ear cover members 19, 21. The amplifier circuit 54 has a microphone input 58 in electrical connection with the microphone 46, speaker output 60 in electrical connection with the speaker 50, and an amplifier volume control input 62 in electrical connection with the volume adjustment knob 56. The stethoscope-coupling cavity 36 is acoustically isolated from the speaker cavity 42 by a portion of the ear cover member.

In the alternative embodiment, the left and right ear assemblies 18,20 are of substantially identical in construction being mirror images of each other. Left and right ear assemblies 18,20 each include an amplifier assembly 32. Sound insulator 30 is molded from an acoustic insulating foam material into a bowl shape having an ear receiving cavity 34. The sound insulator 30 also defines a microphone coupling cavity 36 formed into an exterior facing side 38, amplifier circuit cavity 40 formed entirely therein, and speaker cavity 42 formed in the interior surface 44 and in acoustical communication with the ear receiving cavity 34.

The instant invention has been shown and described herein in what is considered to be the most practical and preferred embodiment. It is recognized, however, that departures may be made therefrom within the scope of the invention and that obvious structural and/or functional modifications will occur to a person skilled in the art.

What is claimed is:

1. A stethoscope sound isolation headset assembly for receiving and isolating sound detected by the stethoscope and transferring it to a user's ears, said assembly comprising:
at least one headband;
a left ear assembly coupled to a first end of said headband;
a right ear assembly coupled to a second end of said headband;
each of said left and right ear assemblies comprising:
a bowl shaped ear cover member having an interior surface and an exterior surface;
an ear receiving cavity defined by said interior surface, said ear receiving cavity defining a volume that receives, isolates and transfers to the user's ears sound detected by the stethoscope;
stethoscope coupling aperture for receiving a stethoscope's ear adapters;
at least one acoustic insulator secured in said ear receiving cavity and having properties for blocking ambient sound from entering said ear receiving cavity, said acoustic insulator being flexible and resilient, said acoustic insulator having a self-sealing slit in alignment with said stethoscope coupling aperture for snugly receiving and supporting a stethoscope; and
means for securing said acoustic insulator in said ear receiving cavity; and
means for securing said headband to said ear cover members.

2. An assembly as recited in claim 1, wherein said means for securing said headband comprises:
a headband support adaptor rotatably mounted to each of said ear cover members, said headband support adaptors each having a sleeve for receiving the ends of said headband and slidably adjusting the height of said headband.

3. An assembly as recited in claim 2, wherein said headband support adaptors each comprise tapered crescent shaped side edges for facilitating secure gripping of said adaptors when adjusting said headband.

4. An assembly as recited in claim 3, wherein said headband support adaptors are adapted for securing said headband with friction.

5. An assembly as recited in claim 2, wherein said headband comprises a rigid arcuate band having a first end and a second end, said first and second ends having a width that is at least substantially equal to the width of said sleeves.

6. An assembly as recited in claim 5, wherein said headband comprises a slot having a plurality of crescent shaped grooves and wherein said sleeves have an interiorly disposed tab for engaging said crescent shaped grooves to secure said headband in a desired location.

7. An assembly as recited in claim 2, wherein said adaptor sleeve comprises shaft that penetrates said ear cover members for facilitating rotational movement.

8. An assembly as recited in claim 1, wherein said at least one headband comprises:
a rigid arcuate headband rotatably and removably mounted at opposite ends to said ear cover members; and
a flexible, length adjustable headband secured at opposite ends to said ear cover members.

9. An assembly as recited in claim 1, further comprising a rigid support plate in each ear receiving cavity for securing said acoustic insulator in said ear receiving cavities and providing mechanical support for a stethoscope, said rigid support plate having an aperture in alignment with said self-sealing slit for receiving the stethoscope and being secured to an interior surface of said ear cover member.

10. An assembly as recited in claim 1, wherein said at least one acoustic insulator comprises:
a first acoustic insulator and a second acoustic insulator secured in said ear receiving cavity in contiguous relationship for enhancing sound barrier characteristics.

11. An assembly as recited in claim 1, wherein said at least one acoustic insulator comprises a rubber-based material.

12. An assembly as recited in claim 10, wherein said first acoustic insulator comprises a rubber-based material and said second acoustic insulator comprises a foam-based material.

13. An assembly as recited in claim 1, wherein said headband comprises a flexible, length adjustable headband secured at opposite ends to said ear cover members.

14. An assembly as recited in claim 13, wherein said headband comprises a plurality of apertures and at least one tab for adjusting the length of said headband.

15. An assembly as recited in claim 1, further comprising:
a flexible, resilient gasket disposed around a circumferential edge of each of said ear cover members for sealing said left and right ear assemblies against a user's head so as to substantially seal potential acoustic leaks.

16. An assembly as recited in claim 1, wherein said ear cover members are molded from an acoustic insulating material into a bowl shape so as to define said ear receiving cavity, said acoustic insulating material defining said stethoscope coupling cavity and a smaller speaker cavity in said ear receiving cavity for projecting sounds to the user's ears.

17. An assembly as recited in claim 1, further comprising:
an amplifier assembly including an amplifier circuit positioned within an amplifier cavity defined by said acoustic insulator inside said ear receiving cavity, a microphone having an acoustical input in connection with said stethoscope coupling cavity, a speaker installed within a speaker cavity defined by said acoustic insulator inside said ear receiving cavity, said speaker being positioned in acoustical communication with said ear receiving cavity, and a volume adjustment knob moveably positionable and mounted to said ear cover member.

18. An assembly as recited in claim 17, wherein said amplifier circuit further has a microphone input in electrical connection with said microphone, a speaker output in electrical connection with said speaker, and an amplifier volume control input in electrical connection with said volume adjustment knob.

19. An assembly as recited in claim 17, further comprising:
means, disposed in said ear receiving cavity, for amplifying sound received from a stethoscope.

20. An assembly as recited in claim 1, further comprising a stethoscope.
